(19) United States
(12) Patent Application Publication
(10) Pub. No.: US 2006/0136069 A1
(43) Pub. Date: Jun. 22, 2006

(54) Device and method for prosthetic implant measurement

(76) Inventor: Antonio Luiz Francalacci Franca, Curitiba (BR)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 11/305,227

(22) Filed: Dec. 19, 2005

(30) Foreign Application Priority Data
Dec. 20, 2004 (BR) MU8403128-0

(51) Int. Cl. A61F 2/04; A61F 2/12

(52) U.S. Cl. 623/23.67; 623/8; 33/512

(57) ABSTRACT

A prosthetic implant measurement device includes a template adapted to fit over an implant site and shaped to conform to a desired post-surgical result and an expandable bladder adapted for insertion into the implant site. The template has at least two holes for passing a catheter attached at one end to the bladder from within the implant site to a supply of fluid for filling the bladder and expanding the implant site. A method of measuring a prosthetic implant includes inserting the bladder into the implant site, passing the catheter through a catheter hole, and fitting the template to the patient at the implant site. The bladder is then filled until the implant site conforms to the template. The volume of fluid required to so fill the bladder corresponds to the volume of the prosthesis required to achieve the desired surgical outcome.

### CROSS-REFERENCE TO RELATED APPLICATIONS

This application claims priority under 35 U.S.C. § 119 to Brazilian Patent Application No. MU8403128-0, filed Dec. 20, 2004, the disclosure of which is hereby incorporated by reference in its entirety.

### FIELD OF THE INVENTION

The present invention relates generally to cosmetic surgery. More particularly, the present invention relates to accurate measurement of prosthetic implants, such as breast implants.

### BACKGROUND OF THE INVENTION

Surgical implantation of material to enhance appearance has become relatively commonplace for both reconstructive and cosmetic purposes. In reconstructive surgery, the surgeon has relative autonomy to size and shape the implant according to professional standards. For cosmetic surgery, however, where the surgeon is hired to perform a definite task and achieve particular results, a successful surgical outcome depends on factors beyond the physician's technical knowledge and skill.

A popular form of such an enhancement procedure is breast augmentation surgery. In cosmetic breast augmentation surgery, it is necessary to clearly and accurately define the patient's desires regarding post-operative breast size and shape. Once the desired surgical result is determined, it is necessary to calculate the implant size required to enlarge the breast from its current size to the desired size.

There are a number of extant pre-operative methods for determining the desired breast size and shape, and the corresponding required implant size, including subjective evaluation, the use of prosthetic mock-ups, computer modeling, and tri-dimensional photography. U.S. Pat. No. 6,796,875 to Placik (“Placik”), for example, teaches the use of resilient, fluid-filled bladders inserted into brassiere-like garments. The patient can inflate or deflate the bladders to achieve the desired breast size and report the volume of fluid necessary to reach the desired result to the surgeon. The surgeon uses the volumes reported by the patient to prepare implants.

Other prior art methods measure the volume of the required implants during the surgery. This may be accomplished, for example, by inserting an elastomeric balloon into the patient's breast and measuring the amount of fluid injected into the balloon necessary to achieve the desired breast size. Once the physician determines that the desired breast size has been achieved, the fluid and balloon are removed and replaced by a prosthesis of volume corresponding to that of the fluid injected into the balloon. Such a method is described in U.S. Pat. No. 4,624,671 to Kress (“Kress”).

Prior art methods are, however, inexact, and do not permit accurate verification of the required implant size during surgery, such that the end result may not closely conform to the patient's pre-operative wishes. Thus, it is not uncommon for the patient to be unhappy with the result, believing that the post-operative breasts are smaller or larger than the pre-operative agreement. For example, the resultant breast size and shape according to Placik will be known only after surgery. Further, since Placik suggests that the patient measure the volume of the desired implant in private and report that volume to the surgeon, it is difficult for the surgeon to objectively verify the patient's measurements and ensure that the post-surgical results closely conform to the patient's desires. Similarly, in Kress, the final volume of the prosthesis is set by the surgeon's subjective visual and manual inspection of the breast during the balloon-inflation phase of the surgery, and may not closely conform to the pre-surgical agreement. Moreover, extant devices used to measure the required volume of the prostheses do not lend themselves to objective verification that the post-operative breast closely conforms to the pre-operative agreement.

Accordingly, it is desirable to provide a device that can be used to objectively establish the desired size of the breast during the pre-surgical phase, to accurately measure the required volume of the breast prostheses during surgery, and to objectively verify that the post-operative breast conforms to the pre-operative agreement.

### SUMMARY OF THE INVENTION

The foregoing needs are met, to a great extent, by the present invention, wherein in one aspect an apparatus is provided that in some embodiments can be used to identify the desired size of the post-operative breast, to measure the volume of the implant required to achieve the desired post-operative breast size, and to verify that the post-operative breast size corresponds to the patient's desires.

In accordance with one embodiment of the present invention, a prosthetic implant measurement device is provided having a template adapted to fit over a prosthetic implant site during surgery. The shape of the template corresponds to the desired post-surgical outcome, so as to permit accurate determination of the implant size required to achieve the desired post-surgical result. The template has at least two catheter holes therethrough, located and adapted to pass a catheter attached to an expandable bladder inside the surgical site and a fluid supply without disturbing the interface between the template and the patient's body. In embodiments, the template is marked to aid in identification and positioning, and may be transparent to further aid in positioning the template on the patient.

In accordance with another aspect of the present invention, a method of preparing and verifying a properly sized prosthetic implant, such as a breast implant, is provided. The method includes providing a shaped template corresponding to the desired post-surgical outcome (i.e., breast size) and an expandable bladder, inserting the bladder into an implant site (i.e., the breast to be enlarged), positing the template over the implant site (i.e., over the patient's breast, and measuring the volume of fluid required to fill the bladder until the implant site conforms to the template. A prosthesis having a volume corresponding to the thus-measured volume may then be implanted. The template may also be used to aid in identification of the desired post-surgical result and verification of the actual post-surgical result.

In accordance with yet another embodiment of the present invention, there is provided a system for selecting and sizing prosthetic implants. The system includes means for determining a desired surgical outcome, means for verifying that the actual surgical outcome corresponds to the desired surgical outcome, and means for calculating the volume of the implant required to achieve the desired surgical outcome.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a perspective view illustrating a prosthetic implant measurement device according to a preferred embodiment of the invention.

FIG. 2 is a top view of a template according to the present invention.

FIG. 3 is a sectional view of a preferred embodiment of the present invention configured for use in a breast enhancement surgery.

### DETAILED DESCRIPTION

The invention will now be described with reference to the drawing figures, in which like reference numerals refer to like parts throughout. An embodiment in accordance with the present invention provides a device for the measurement of prosthetic implants having a template adapted to fit over an implant site during surgery and shaped to correspond to the desired post-surgical result. At least two catheter holes are provided on the template in order to pass a catheter attached at one end to an expandable bladder from inside the implant site to a fluid supply without disturbing the fit of the template over the surgical site. This permits the expansion of the bladder, and thereby of the patient's body part, until it fully conforms to the template, allowing for an accurate measurement of the volume of the prosthesis required to achieve the desired result. Further, the template may be used without the expandable bladder during the pre-surgical phase to select a desired result, and during the post-surgical phase to verify that the actual surgical result corresponds to the desired surgical result.

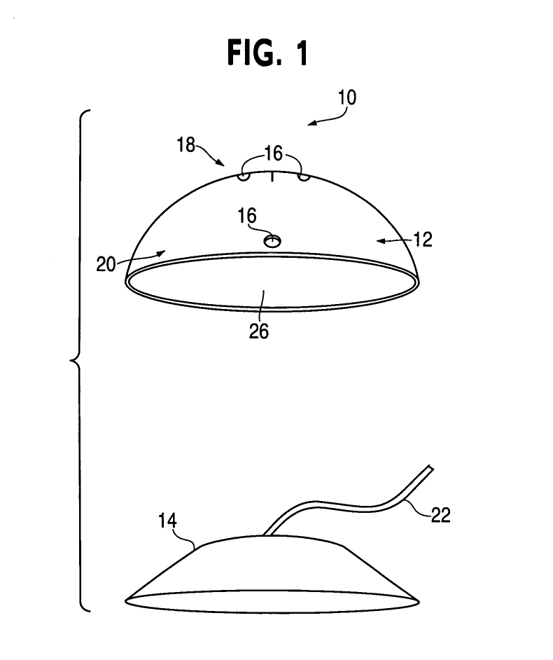

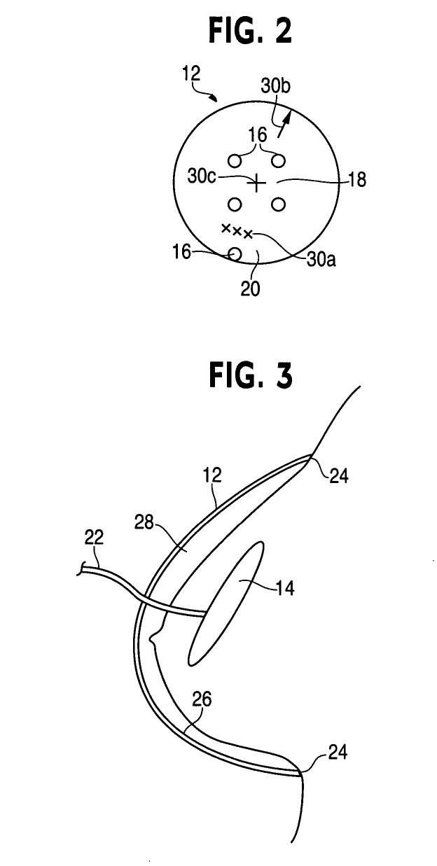

An embodiment of the present inventive apparatus is illustrated in FIG. 1. A prosthetic implant measurement device 10 generally includes a template 12 and an expandable internal bladder 14 adapted for insertion into a prosthetic implant site (that is, within a dissected region into which the prosthetic implant will be placed). Template 12 is adapted to fit over the prosthetic implant site during surgery. The shape of template 12 conforms to a desired surgical outcome. For example, as illustrated in FIGS. 1 through 3 and described herein, template 12 may be adapted for use in breast augmentation surgery. In this embodiment, template 12 will have a domed shape generally conforming to the shape of a human breast, and will be sized to conform to the patient's desired augmented, post-surgical breast.

At least two catheter holes 16 are provided through template 12. In embodiments, template 12 includes five catheter holes 16 therethrough. Four catheter holes 16 are provided on an upper portion 18, generally corresponding to the areola, while a fifth catheter hole 16 is provided on a side portion 20. Catheter holes 16 on upper portion 18 may be symmetrically arranged. Catheter holes 16 are of sufficient diameter to pass a catheter tube 22, and are located to pass catheter tube 22 without disturbing an interface 24 between template 12 and the prosthetic implant site. That is, catheter holes 16 permit proper accommodation of template 12 over the patient's breast, which in turn permits proper conforming of the enlarged breast to the underside 26 of template 12, which results in an accurate determination of the size of the required prosthetic implant.

One end of catheter tube 22 is attached to bladder 14 and is adapted to fill bladder 14 with a fluid, such as saline. In certain embodiments, this is accomplished by attaching the free end of catheter tube 22 to a syringe. As bladder 14 is filled with the fluid, the breast will gradually expand, filling interstitial volume 28, until it conforms to underside 26 of template 12. The volume of fluid required to fill bladder 14 to this point corresponds to the size of the prosthetic implant required to achieve the desired post-surgical result.

In embodiments, template 12 includes orientation markings 30. Orientation markings 30 may be used to identify the size of template 12 or to properly orient template 12 on the patient. For example, orientation markings 30 may include a legend 30a describing the size of template 12 in relation to brassiere sizes or breast volume, an arrow 30b used to properly position template 12 on the breast, and a hash mark 30c corresponding to the nipple. To aid in positioning template 12 on the prosthetic implant site using orientation markings 30, template 12 may be transparent.

The use of measurement device 10 to perform a breast augmentation, or mammoplasty, will now be described. Mammoplasty can be divided into five general phases: identification of current breast size, choice of new breast size, estimation of probable prosthetic implant volume, verification of actual prosthetic implant volume, and confirmation of surgical results.

Identification of Current Breast Size. Templates 12 may be used to identify the current breast size. To speed this process, the first test may be conducted with template 12 corresponding to the patient's pre-surgical brassiere size. If the pre-surgical breast does not conform to this template 12, then template 12 is too large, and a smaller template 12 should be tested. Similarly, if the pre-surgical breast spills forth from this template 12, then template 12 is too small, and a larger template 12 should be tested. The pre-surgical breast size is accurately identified when template 12 is completely filled by the breast without any exceeding tissue. The surgeon will take note of the size of template 12.

Choice of New Breast Size. Larger templates 12 are then fitted to the patient. To aid in visualization of the post-surgical result, the use of a light-colored, skin-tight blouse is advised. The patient chooses template 12 conforming to the desired post-surgical outcome, and the surgeon takes note of the size of the selected template 12.

Estimation of Probable Prosthetic Implant Volume. Once the current breast size and desired post-surgical outcome size are known, the surgeon can estimate the probable volume of the required prosthetic implant. This may be accomplished, for example, by using a table crossing current breast size with desired breast size.

Verification of Actual Prosthetic Implant Volume. This step occurs during the surgical procedure. An incision is made, the implant site is dissected, and bladder 14 is inserted therein. Catheter tube 22 passes through the incision, through catheter hole 16 in template 12 corresponding to the desired surgical outcome, and is attached to a fluid supply. Template 12 is situated over the breast, such as illustrated in FIG. 3, according to orientation markings 30 thereon. Since catheter tube 22 passes through catheter hole 16, template 12 may properly accommodate over the patient's breast without interference at interface 24. Bladder 14 is then filled until the breast conforms to underside 26 of template 12, and the surgeon measures the volume of fluid required to reach this state. Bladder 14 is emptied and removed, and both template 12 and bladder 14 are discarded. A prosthetic implant having a volume corresponding to that measured is then prepared and implanted.

Confirmation of Surgical Results. Once the healing process is substantially complete, approximately six months after the surgery, the surgical result is confirmed by positioning template 12 corresponding to the desired surgical outcome over the patient's breast. A successful surgical outcome is confirmed where template 12 fits properly over the breast (that is, where the breast conforms to template 12 without any exceeding tissue). In this way, any patient doubts that the breast is smaller or larger than originally agreed upon can be removed.

Although an example of measurement device 10 is illustrated and described in the context of breast augmentation surgery, it will be appreciated that measurement device 10 may be used for any prosthetic implant surgery, such as calf implants or pectoral implants. The shape of template 12 will depend on the intended surgical application of measurement device 10.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A prosthetic implant measurement device, comprising:
a template adapted to fit over a prosthetic implant site during surgery and having a shape conforming to a desired surgical outcome, said template having at least two catheter holes therethrough.

2. The measurement device of claim 1, further comprising:
an expandable internal bladder adapted for insertion into the prosthetic implant site; and
at least one catheter tube attached to said bladder, said catheter sized to pass through said catheter hole and adapted to fill said bladder with a fluid.

3. The measurement device of claim 2, wherein said bladder is filled with the fluid until the prosthetic implant site conforms to said template.

4. The measurement device of claim 3, wherein a filled volume of said bladder is used to size a prosthetic implant.

5. The measurement device of claim 1, wherein said at least two catheter holes permit passage of a catheter from within the prosthetic implant site without disturbing an interface between said template and the prosthetic implant site.

6. The measurement device of claim 5, wherein at least one catheter hole is located on an upper portion of said template, and wherein at least one catheter hole is located on a side portion of said template.

7. The measurement device of claim 1, wherein said measurement device comprises a breast implant measurement device, and wherein the prosthetic implant site is a human breast.

8. The measurement device of claim 1, wherein said template is transparent.

9. The measurement device of claim 1, wherein said at least two catheter holes comprises five catheter holes.

10. The measurement device of claim 1, wherein said template further comprises orientation markings.

11. A method of preparing and verifying a properly sized prosthetic implant, comprising:
providing a template having a shape conforming to a desired surgical outcome and an expandable internal bladder;
inserting the bladder into an implant site;
positioning the template over the implant site; and
measuring a volume of fluid required to fill the bladder until the implant site conforms to the template.

12. The method of claim 11, further comprising implanting a prosthetic implant having a volume corresponding to the volume of fluid measured in said measuring step.

13. The method of claim 11, further comprising identifying the desired surgical outcome.

14. The method of claim 13, wherein identifying the desired surgical outcome comprises selecting, from a plurality of templates, the template having a shape conforming to the desired surgical outcome.

15. The method of claim 14, further comprising estimating a required implant size based on the selected template.

16. The method of claim 11, further comprising verifying correspondence between an actual surgical outcome and the desired surgical outcome after healing is substantially complete.

17. The method of claim 16, wherein verifying comprises comparing the template to the actual surgical outcome.

18. A prosthetic implant selection and sizing system, comprising:
means for determining a desired surgical outcome and verifying correspondence between an actual surgical outcome and the desired surgical outcome; and
means for calculating a volume of a prosthetic implant required to achieve the desired surgical outcome.

19. The system of claim 18, wherein said calculating means comprises:
a template adapted to fit over a prosthetic implant site during surgery and having a shape corresponding to the desired surgical outcome; and
means for enlarging the prosthetic implant site until the prosthetic implant site conforms to said template.

20. The system of claim 19, wherein said enlarging means comprises an expansible bladder inserted into the prosthetic implant site and supplied with a fluid.
